(19) United States
(12) Patent Application Publication
(10) Pub. No.: US 2014/0009739 A1
(43) Pub. Date: Jan. 9, 2014

(54) WEARABLE APPARATUS AND IR CONFIGURATION FOR OPTIMIZING EYE-TRACKING USED FOR HUMAN COMPUTER INTERACTION

(71) Applicant: Lindsay Greco, (US)

(72) Inventors: Devon Greco, Ambler, PA (US); Domenic Greco, Fort Washington, PA (US)

(21) Appl. No.: 13/918,896

(22) Filed: Jun. 14, 2013

Related U.S. Application Data
(60) Provisional application No. 61/659,919, filed on Jun. 14, 2012.

(51) Int. Cl. A61B 3/113

(52) U.S. Cl. CPC A61B 3/113
USPC 351/206

(57) ABSTRACT

A highly portable wearable apparatus is provided to capture high-quality image data of eye movement used for eye-tracking. The wearable apparatus comprises a coupling member adapted to securely couple the apparatus to an eyewear frame, an arm assembly having a distal end and configured to enable vertical and lateral of movements of the distal end, and an imaging assembly disposed on the distal end. The imaging assembly, via vertical and lateral movements of the distal end, can be disposed in a position close to an eye of a user that is suitable for eye-tracking when the user wears an eyewear frame with the apparatus deployed thereon. The imaging assembly is configured to capture infrared-illuminated eye and wirelessly transmit captured image data. A configuration in connection with infrared source placement is provided to achieve optimal glint-tracking used to track head movement for improving quality of eye-tracking.

### CROSS-REFERENCE TO RELATED APPLICATION

This application claims the benefit under 35 U.S.C. §119(e) of Provisional Patent Application No. 61/659,919, filed Jun. 14, 2012, the entire disclosure of which is hereby incorporated by reference.

### BACKGROUND

1. Technical Field

The present disclosure generally relates to one or more apparatuses and methods used for human computer interaction (HCI). More particularly, the present disclosure relates to a highly portable and effective wearable apparatus adapted to track movement of an eye in close range and wirelessly transmit data relating to or derived from tracking of eye movement to a receiving device, such as a computer with which HCI is realized.

2. Description of the Related Art

Communication and interaction with computers has become essential for our modern daily lives. HCI systems based on interface devices such as keyboards, mice, touch-screen, and touch pads have many limitations such as accuracy, ergonomics, portability, and multi-tasking. The shortcomings of these HCI systems prevent users from interacting with technology to its full potential.

Conventional HCI systems based on eye tracking are configured for Augmentative and Alternative Communication (AAC), for use by individuals with physical disabilities. These systems are often built into proprietary hardware platforms that are bulky, expensive, and unreliable. For example, there are available HCI systems using eye-tracking devices that are known as “remote trackers”, which are modular units that attach to a video display system and track eye movement (e.g., pupil movement) at a distance or “remotely”. These units are not portable and the distance from a user's eyes to the tracking device needs to be precise. Thus, to Applicant's knowledge, for such HCI systems, portability is a major issue.

Also, those HCI systems are known to have low tolerance to user movement (such as head movement) during eye-tracking. Thus, movement of a user disturbs the accuracy of HCI. This known weakness greatly inhibits a user's ability to multi-task during eye-tracking, and thus greatly reduces the usability of those systems.

Additionally, there have been available eye-tracking software programs for processing received eye-tracking data and translating tracked eye movements (e.g., pupil movements and eyelid movements) into corresponding cursor movements or cursor clicks on a computer screen. The effectiveness of such software programs heavily relies on the quality of eye-movement-related image data captured by hardware devices used for eye-tracking. However, to Applicant's knowledge, there have not been available readily portable and highly eye-tracking-effective hardware devices that can be “hooked up” with those available eye-tracking software programs to achieve optimal HCI results.

Thus, there is a need for hardware devices which are easily portable and capable of producing high-quality and highly effective eye-tracking data that can be used by one or more available eye-tracking software programs to form an optimal HCI system.

### BRIEF SUMMARY

In one aspect, the present disclosure provides a highly portable wearable apparatus adapted to deploy an imaging device (for capturing eye movement images) in a position close to a subject eye of a user and suitable for eye-tracking, produce high-quality eye-tracking data, and wirelessly transmit produced eye-tracking data to a receiving device equipped with one or more eye-tracking software programs.

In another aspect, the present disclosure provides an eye-tracking imaging assembly capable of capturing high-quality images of eye movements of a subject eye illuminated by infrared light and transmitting acquired eye-tracking data to a receiving device equipped with one or more eye-tracking software programs.

In yet another aspect, the present disclosure provides a configuration adapted to not only achieve high-quality infrared illumination on an subject eye, but also create a clearly visible glint that can be used by an eye-tracking software program as a static reference point for tracking head movement so as to enhance the quality of eye-tracking and produce more accurate and effective HCI results.

The above summary contains simplifications, generalizations and omissions of detail and is not intended as a comprehensive description of the claimed subject matter but, rather, is intended to provide a brief overview of some of the functionality associated therewith. Other systems, methods, functionality, features and advantages of the claimed subject matter will be or will become apparent to one with skill in the art upon examination of the following figures and detailed written description.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

FIGS. 1A-E are pictorials depicting an exemplary wearable apparatus configured for high-quality eye-tracking, according to one or more embodiments of the present disclosure. FIGS. 1A and 1B are two pictorials illustrating an exemplary wearable apparatus from two different perspectives, according to one or more embodiments of the present disclosure. FIGS. 1C and 1D are two pictorials illustrating a headset of an exemplary wearable apparatus headset when the headset is in “folded” and “unfolded” configurations, respectively, according to one or more embodiments of the present disclosure. FIG. 1E is a pictorial illustrating a user managing to have an eye-tracking image assembly disposed at an exemplary position optimal for eye-tracking through wearing an exemplary wearable apparatus disclosed in the present disclosure, according to one or more embodiments of the present disclosure.

FIG. 2 is a diagram illustrating functional modules of an eye-tracking imaging assembly, according to one or more embodiments of the present disclosure.

FIGS. 3A-D are diagrams and pictorials which illustrate a novel configuration in connection with an infrared illumination device, a configuration which enhances the quality of eye-tracking in an HCI system, according to one or more embodiments of the present disclosure. FIG. 3A is a diagram illustrating a relative positioning configuration between an infrared illumination device and a monitor displaying cursor movements, according to one or more embodiments of the present disclosure. FIGS. 3B and 3C are two pictorials illustrating an example of the exemplary configuration from two different perspectives, according to one or more embodiments of the present disclosure. FIG. 3D is a diagram illustrating a relative positioning of a glint created as a result of the exemplary configuration, according to one or more embodiments of the present disclosure.

### DETAILED DESCRIPTION

In the following detailed description of exemplary embodiments of the disclosure, specific exemplary embodiments in which the disclosure may be practiced are described in sufficient detail to enable those skilled in the art to practice the disclosed embodiments. For example, specific details such as specific method orders, structures, elements, and connections have been presented herein. However, it is to be understood that the specific details presented need not be utilized to practice embodiments of the present disclosure. The following detailed description is, therefore, not to be taken in a limiting sense, and the scope of the present disclosure is defined by the appended claims and equivalents thereof.

References within the specification to “one embodiment,” “an embodiment,” “embodiments”, or “one or more embodiments” are intended to indicate that a particular feature, structure, or characteristic described in connection with the embodiment is included in at least one embodiment of the present disclosure. The appearance of such phrases in various places within the specification are not necessarily all referring to the same embodiment, nor are separate or alternative embodiments mutually exclusive of other embodiments. Further, various features are described which may be exhibited by some embodiments and not by others. Similarly, various requirements are described which may be requirements for some embodiments but not other embodiments.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. Moreover, the use of the terms first, second, etc. do not denote any order or importance, but rather the terms first, second, etc. are used to distinguish one element from another.

Within the descriptions of the different views of the figures, the use of the same reference numerals and/or symbols in different drawings indicates similar or identical items, and similar elements can be provided similar names and reference numerals throughout the figure(s). If a reference numeral is once used to refer to a plurality of like elements, unless required otherwise by context, the reference numeral may refer to any, a subset of, or all of, the like elements in the figures bearing that reference numeral. The specific identifiers/names and reference numerals assigned to the elements are provided solely to aid in the description and are not meant to imply any limitations (structural or functional or otherwise) on the described embodiments.

Functional steps illustrated herein, unless logically required to be performed in accordance with a specific sequence, are presumed to be performable in any order without regard to a specific sequence.

In the description, relative terms such as “left,” “right,” “vertical,” “horizontal,” “upper,” “lower,” “top” and “bottom” as well as any derivatives thereof (e.g., “left side,” “lower arm,” etc.) should be construed to refer to the logical orientation as then described or as shown in the drawing figure under discussion. These relative terms are for convenience of description and are not intended to convey any limitation with regard to a particular orientation.

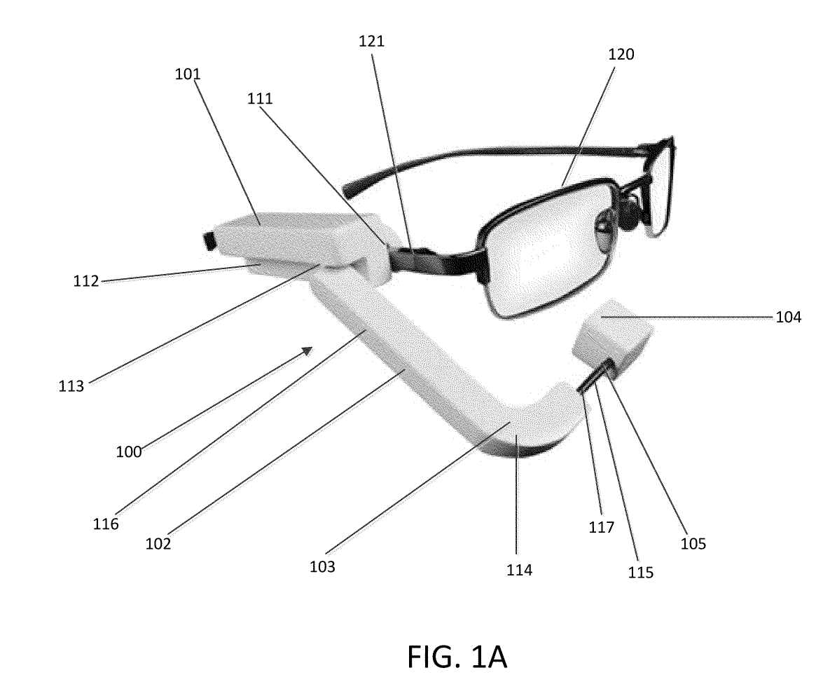

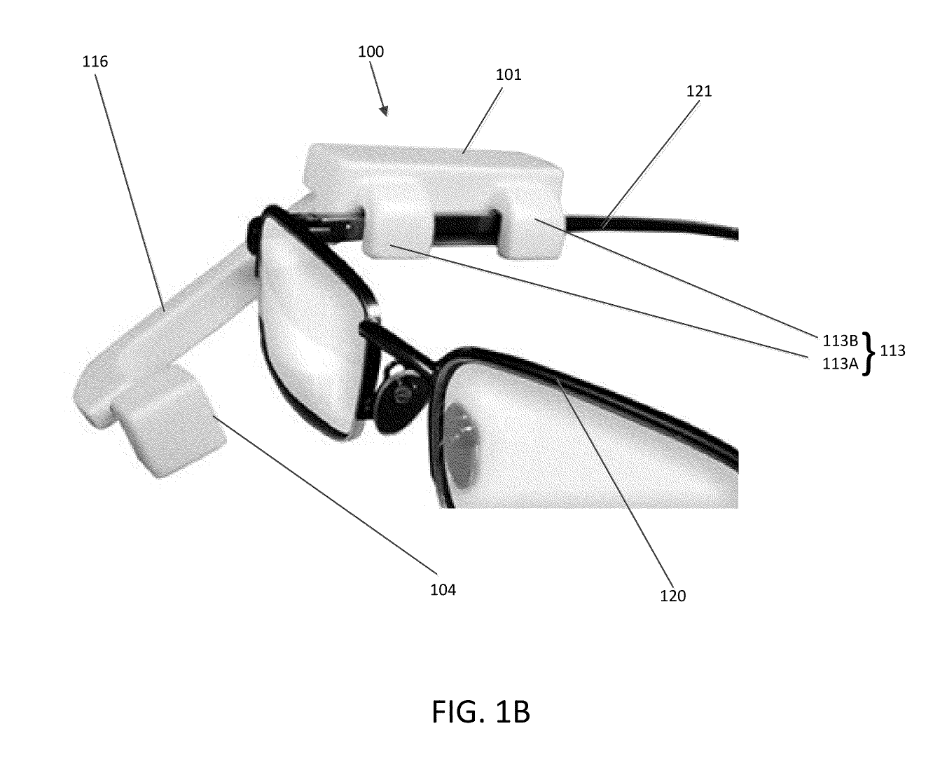

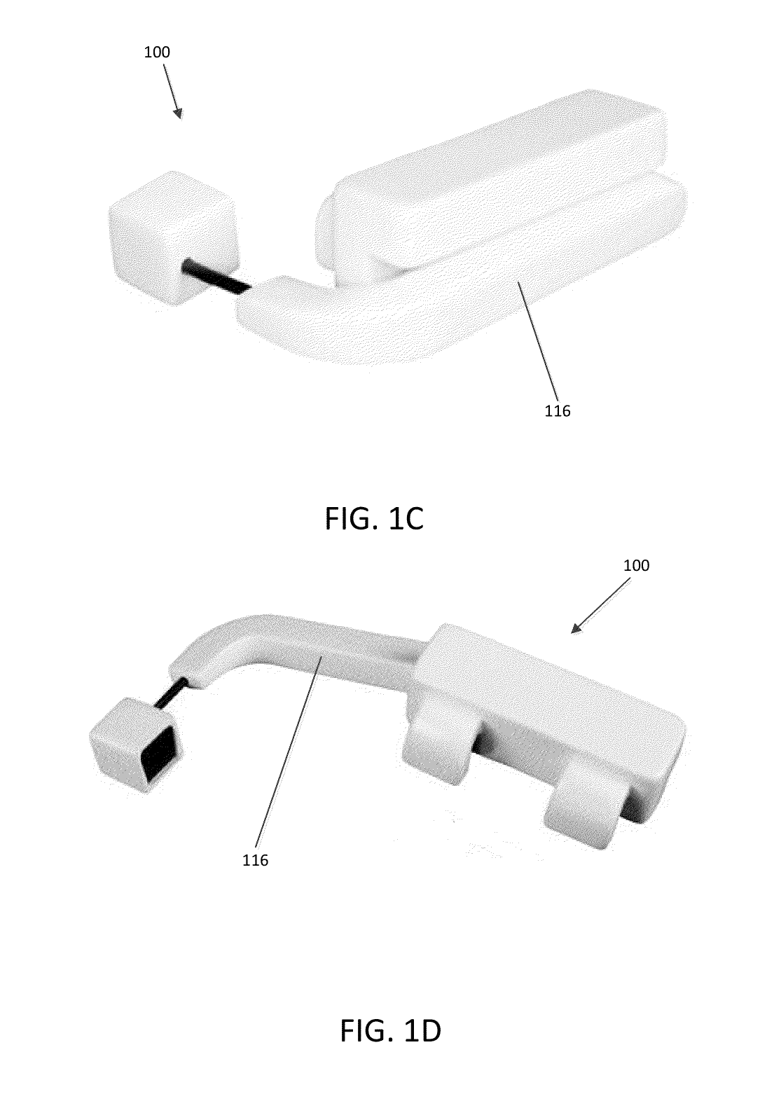

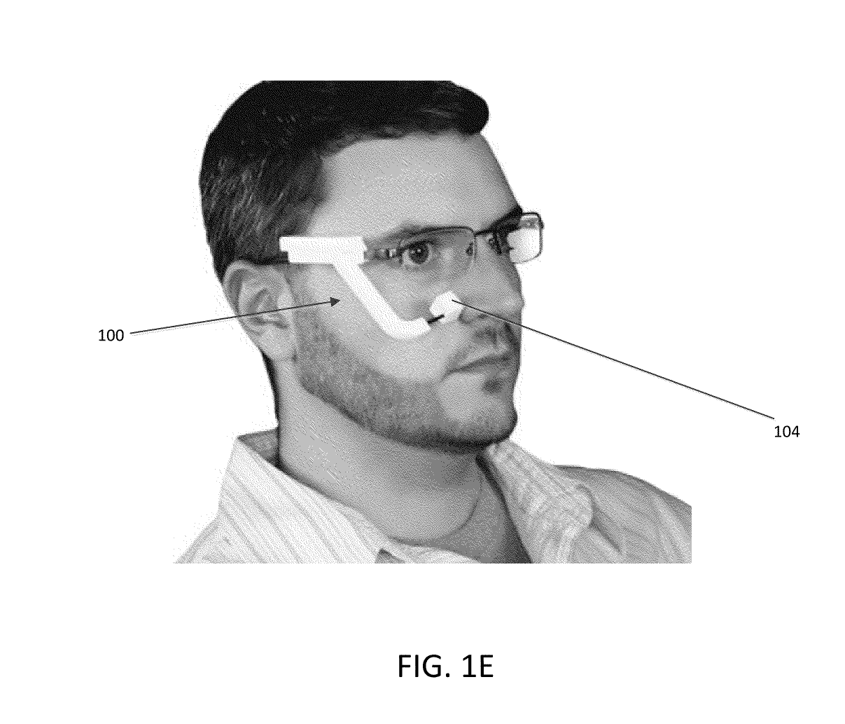

With reference now to the figures, and beginning with FIGS. 1A-D, there are depicted an exemplary wearable apparatus configured for high-quality eye-tracking, according to one or more embodiments of the present disclosure.

Referring to FIGS. 1A and 1B, which depict the wearable apparatus from two different angles, the illustrated wearable apparatus is provided in the form of a wearable “headset” 100 coupled to one side arm 121 of an eyewear frame 120. As used herein, the terms “eyewear frame” and “eyewear” may be used interchangeably, when allowed by the context where either is used, to refer to the physical frame of an eyewear, an eyewear as a wearable object, or both. The headset includes a coupling member 101, which is configured and structured to enable the headset to be securely coupled to side arm 121 of eyewear frame 120 when the headset is deployed on eyewear frame 120. The coupling member 101 comprises a clip 111 configured and structured to mount headset 100 onto side arm 121 of eyewear frame 120 such that the headset is securely fastened to eyewear frame 120.

In one embodiment, clip 111 comprises side members 112 and 113 configured and structured to tightly retain side arm 121 of eyewear frame 120 there-between. In one implementation, side members 112 and 113 are joint at one or more pivots and spring-biased (not shown) against each other, such that when side arm 121 of eyewear frame 120 is disposed there-between, opposing biasing forces there-between exerted against both sides of side arm 121 result in side arm 121 being securely retained there-between. Other biasing means may be used in place of or in addition to the aforementioned spring-biasing means. Side member 113 may comprise two retaining parts 113A and 113B configured and structured to securely retain a side arm of eyewear frame 120 against the other side member 112.

In one embodiment, headset 100 further includes an arm assembly 102 and an eye-tracking imaging assembly 104, with eye-tracking imaging assembly 104 disposed on or near a distal end 105 of arm assembly 102. Eye-tracking imaging assembly 104 is detachably or indetachably coupled to distal end 105 of arm assembly 102. Arm assembly 102 is structured and configured to let eye-tracking imaging assembly 104 (disposed on or near distal end 105 of arm assembly 102) move vertically as well as laterally, thereby enabling eye-tracking imaging assembly 104 to be adjustably positioned close to (e.g. slightly below) the center of an eye of a user for a clear view of, e.g., the cornea and pupil thereof when eyewear frame 120 is worn by the user with headset 100 deployed thereon.

In one embodiment, arm assembly 102 includes an L-shaped lever arm unit 103 whose one end 113 of its arm member 116 is pivotably fastened to side member 112 of clip 111 such that pivotal movements of lever arm unit 103 results in eye-tracking imaging assembly 104 (disposed at distal end 105 of arm assembly 102) moving upward or downward relative to the frame of eyewear 120. Lever arm unit 103 further includes a linkage member 115 connecting the non-pivotal end 114 of arm member 116 of lever arm unit 103 and distal end 105 of arm assembly 102. Thus, in this embodiment, distal end 105 of arm assembly 102 is also the distal end of the lever arm unit 103 and linkage member 115.

As shown, arm member 116 and linkage member 115 form an L shape. In one embodiment, a proximal end of linkage member 115 is detachably attached or joined to arm member 116 at the non-pivotal end 114 of arm member 116. In another embodiment, linkage member 115 and arm member 116 is manufactured as one piece and joined at the non-pivotal end 114 of arm member 116. Thus, hereinafter, for the ease of discussion, the non-pivotal end of arm member 116 and proximal end of linkage member 115 may each be referred to, or collectively referred to, as joint 114.

In one embodiment, a distal portion of linkage member 115 is retractable and extendable such that the distal end of linkage member 115 can move between a fully retracted position (not shown) and a fully extended position (not shown). Retracting or extending linkage member 115 results in eye-tracking imaging assembly 104 (disposed at distal end 105) moving laterally between the fully retracted position and the fully extended position. Thus, in this embodiment, eye-tracking imaging assembly 104 may be fixedly disposed on or near the distal end of linkage member 115 while being able to make lateral movements needed for it to be adjustably positioned at a location suitable for eye-tracking.

In one embodiment, eye-tracking imaging assembly 104 is slidably disposed on linkage member 115 such that eye-tracking imaging assembly 104 may move laterally by sliding laterally along linkage member 115 between a proximal location 117 on linkage member 115 (proximal to joint 114) and distal end 105 of linkage member 115. Thus, in this embodiment, the distal portion of linkage member 115 does not have to be (i.e., may be or may not be) retractable or extendable to realize lateral movement of eye-tracking imaging assembly 104.

Combinations of pivotal movements of lever arm unit 103 and lateral movements of eye-tracking imaging assembly 104 enables the eye-tracking imaging assembly to be selectively and optimally positioned close to the center of the eye for a clear view of the cornea and pupil. Thus, as illustrated in FIG. 1E, a user wearing a headset 111 (through wearing an eyewear 120) is able to dispose, through headset 111, eye-tracking imaging assembly 104 at an exemplary position below while close to an eye of the user, a position which is a suitable or optimal for eye-tracking.

Headset 111 is highly portable, largely due to its relative small size and the pivoting structure between arm assembly 102 and coupling member 101. As shown in FIGS. 1A, 1B and 1E, headset 111 is comparable to the size of an eyewear frame 120. Thus, headset 111 is small relative to a human face. FIG. 1C is a pictorial depicting headset 111 when lever arm unit 103 of arm assembly 102 is “folded” (pivoted all the way to its close position), where arm member 116 of arm assembly 102 lies next to, and forms almost zero degree to, side member 112 of coupling member 101. FIG. 1D is a pictorial depicting headset 111 when lever arm unit 103 of arm assembly 102 is “unfolded” (pivoted all the way to its maximum away position), where arm member 116 of arm assembly 102 almost forms 180 degree from side member 112 of coupling member 101. Thus, as illustrated, when lever arm unit 103 of arm assembly 102 is either pivoted all the way to its close position or to its maximum away position, the general dimensions of headset 111 renders headset 111 suitable for storage and carriage. Accordingly, headset 111 is highly portable.

A skilled artisan readily appreciates that many changes can be made to the wearable apparatus depicted in FIGS. 1A-E without departing from the scope and spirit of the present disclosure. As one example, the coupling member of the headset does not have to be the clipping structure (clip 111) illustrated in FIGS. 1A-E. Any coupling structure that securely couples a wearable headset to any part of an eyewear frame may be used. As another example, the illustrated arm assembly 102 may use other combinations of retractable as well as pivotal structures to achieve the same or similar objective of selectively positioning eye-tracking imaging assembly 104 (disposed at or near a location on the arm assembly) at a position close to an eye that is suitable for eye-tracking.

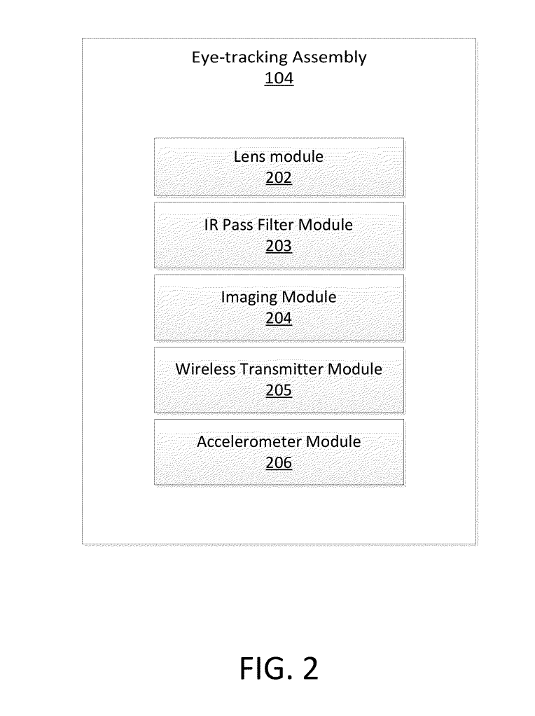

FIG. 2 is diagram illustrating functional modules of an eye-tracking imaging assembly 104, according to one or more embodiments of the present disclosure. Referring to FIG. 2, an eye-tracking imaging assembly 104 comprises an imaging module 204, an infrared (IR) pass filter module 203, and a lens module 202. Imaging module 204 may include an analog imaging module used in an analog camera, a digital imaging module used in a digital camera, or any combination thereof. Any of these analog, digital or combination imaging modules, as well known, is configured and structured to capture static and/or moving images (in the form of image data) as a result of incoming light passing through one or more lens placed in front of an opening thereof. IR pass filter module 203 may include a band pass filter that allows IR light (having, for example, a 750 nm wavelength) to pass. Lens module 202 many include an optical lens tuned to allow or ensure clear focusing on an eye (e.g. its cornea and pupil) situated in close proximity to the eye-tracking imaging assembly 104.

An eye-tracking imaging assembly 104 may further comprise a wireless transmitter module 205 capable of transmitting analog or digital data wirelessly to one or more receiving devices. Wireless transmitter module 205 is communicatively coupled to imaging module 204 such that wireless transmitter module 205 may receive from imaging module 204 image data captured by imaging module 204, data derived from captured image data, and/or any combination thereof, any of which may be used for eye-tracking purposes. Thus, hereinafter, data received from imaging module 204—which may include image data captured by imaging module 204, data derived from captured image data, and/or any combination thereof—will be referred to as “eye-tracking data.” Upon receiving eye-tracking data from imaging module 204, wireless transmitter module 205 may wirelessly transmit the received eye-tracking data to a receiving device (such as a computer with which HCI is realized), which typically runs an eye-tracking software program adapted to translate the eye-tracking data received from imaging module 204 to, for example, corresponding cursor movement(s) and cursor clicks, in realizing HCI.

As illustrated in FIGS. 1A-E, an eye-tracking imaging assembly 104 is configured and structured to be of a small size relative to a human face. In one exemplary implementation, eye-tracking imaging assembly 104 is made from a small-size conventional digital or analog camera equipped with a wireless transmitting device. To make an exemplary eye-tracking imaging assembly 104, starting from the small-size conventional camera, the camera's usually included one or more infrared blocking filters are removed. An infrared pass filter (which blocks visible light and passes infrared light) is then incorporated into the small-size camera in such a manner that the small size of the camera is generally maintained. The small-size camera's one or more lenses are then either re-tuned to a focal length to allow or ensure clear focusing on an eye (including its cornea and pupil) in close proximity, or replaced by one or more new lens having a focal length allowing or ensuring same. If the camera's one or more lenses are replaced, the one or more replacement lenses may be of dimensions comparable to those of the replaced one(s) such that the small size of the modified camera is generally maintained by the replacement operation.

The modified small-size camera thus forms an eye-tracking imaging assembly 104. Hence, with this exemplary implementation, for the newly formed eye-tracking imaging assembly 104, lens module 202 comprises either the re-tuned one or more lenses or the replacement one or more lenses, imaging module 204 comprises the imaging module of the original small-size camera, IR pass filter module comprises the newly incorporated infrared pass filter, and the wireless transmitter module 205 comprises the wireless transmitting device of the original small-size camera.

An eye-tracking imaging assembly 104 may optionally include an accelerometer module 206 communicatively coupled to wireless transmitter module 205. Alternately, an accelerometer module 206 may be provided as part of headset 111 while physically separate from an eye-tracking imaging assembly 104 and communicatively coupled to wireless transmitter module 205 thereof. As well-known, an accelerometer module may measure the rate of change of velocity relative to any inertial frame of reference. Thus, accelerometer module 206 can detect changes in intentional and unintentional head movement(s) of a user during an eye-tracking session.

Accelerometer module 206 may be used in two ways in an Applicant's novel HCI system. First, accelerometer module 206 may be translated to a cursor movement on a computer screen. For, as a user turns his/her head to the left, the accelerometer module detects this head movement, and manages to have the detected head movement information transmitted (wirelessly) to a computer (via wireless transmitter module 205 of an eye-tracking imaging assembly 104). The computer, upon receiving the information indicating the detected head movement, may cause a cursor on a display screen of the computer to move to the left. Similarly, the computer may also respond to received head movement information indicating that the user tilts his/her head up by causing the cursor to move up. Thus, head movement information as detected by the accelerometer module can be used to decide a cursor movement on a computer screen.

Second, accelerometer module 206 may be used for the purpose of implementing detection and/or removal of a movement artifact. In particular, head movement(s) may cause artifact in an eye-tracking operation. For example, as a user stares at the center of a display screen of a computer, the user may turn his/her head right while the user's eyes shift to the left so that the user may remain looking at the same center of the display screen. Applicant's proposed HCI system may register this combination behavior of the user as an intentional gaze in the left direction, which does not reflect the reality that the user continues to stare at the same center of the display screen. Accelerometer module may be used to detect this head movement and partially or fully negate the eye movement (e.g., pupil movement) based on a calculated rate of change in velocity of the head relative to the newly detected intentional stare direction, thus removing part or all of a movement artifact otherwise reflected on an incorrect cursor position on the display screen.

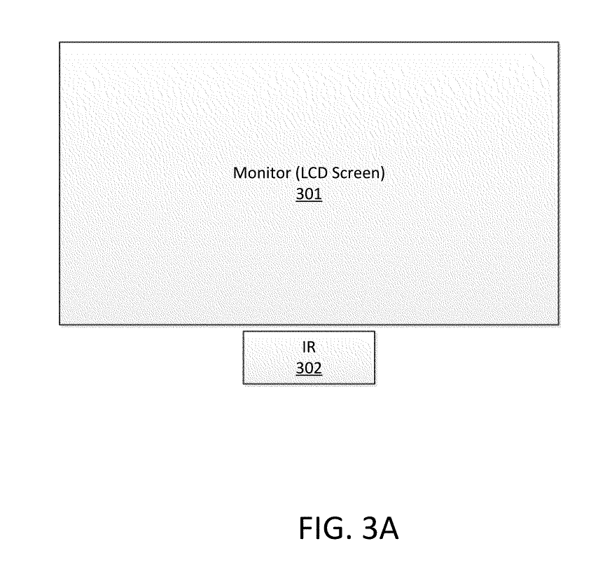

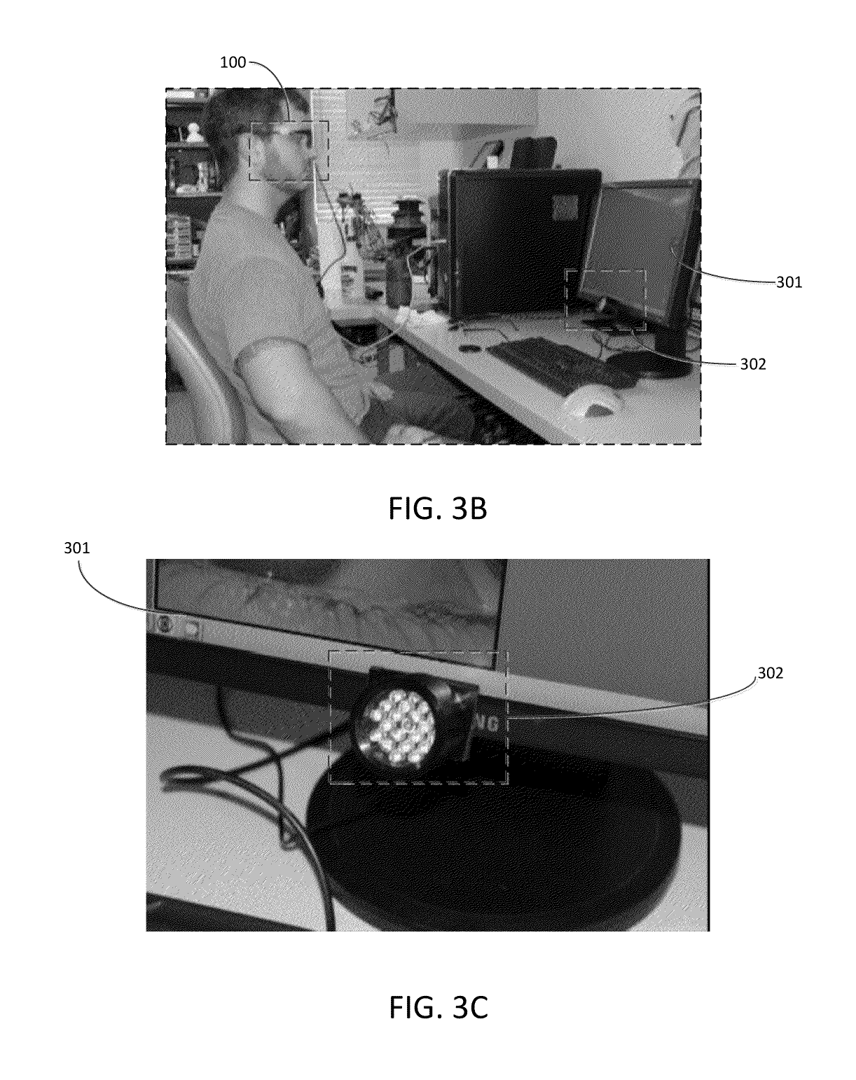

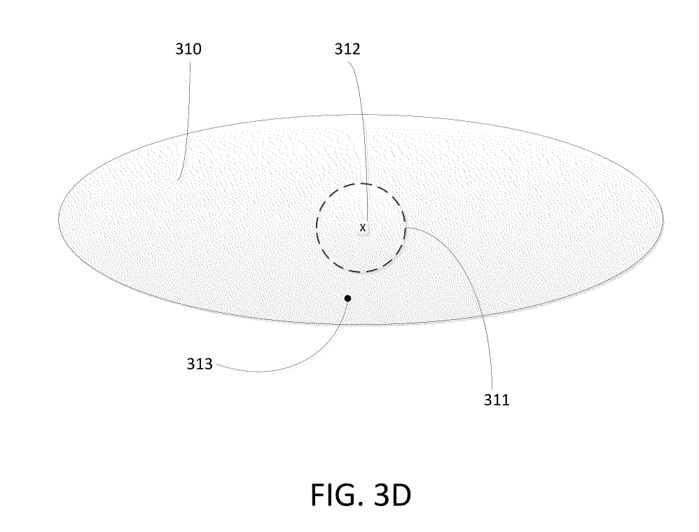

FIGS. 3A-D illustrates a novel configuration in connection with an infrared illumination device which enhances the quality of eye-tracking in an HCI system, according to one or more embodiments of the present disclosure.

As a skilled artisan readily appreciates, the use of infrared light in eye-tracking is well-known. Infrared light has been used in many commercial tracking products for widely understood reasons. First, infrared light produces a high sensitivity to camera light sensor, which increases accuracy. Second, infrared light is invisible to a human eye so it does not distract a user when it is used to illuminate an eye of the user for eye-tracking. Third, infrared can also be filtered from visible light so external light sources such as sunny window or a desk lamp will not interfere with infrared light used during an eye-tracking session. To Applicant's knowledge, the conventional art's use of infrared light in eye-tracking primarily focuses on the illumination aspect of infrared light without exploring some other aspects of infrared light—particularly the aspect in connection with the position of an infrared light source relative to the position of a monitor (e.g., an LCD screen) displaying cursor movements translated from captured eye movements (e.g. pupil movements)—that may enhance the quality of eye-tracking used in an HCI system.

FIG. 3A is a diagram illustrating a relative positioning configuration between an infrared illumination device 302 (hereinafter referred to as “IR device”) and a monitor displaying cursor movements. In one embodiment, IR device 302, which emits infrared light, is positioned at or near the bottom center location of monitor 301. As shown in FIGS. 3B and 3C, which are pictorials illustrating an example of the configuration shown in FIG. 3A, IR device 302 may be configured and structured to fastened to the bottom edge of the monitor at or near the center location of the bottom edge, while facing towards a subject eye of a user which an eye-tracking imaging assembly 104 (disposed in close proximity to the eye using headset 111) conducting image-capturing is facing.

The configuration illustrated in FIGS. 3A-C, in one aspect, results in a subject eye being adequately illuminated by infrared light emitted from IR device 302. Referring to FIG. 3D, since eye-tracking imaging assembly 104 is in close proximity to the subject eye 310 and allows infrared light to pass through, the infrared illumination on subject eye 310 creates a great contrast between the pupil 311 (with the pupil center 312) and the cornea (not shown) of subject eye 310 in images captured by imaging module 204 of eye-tracking imaging assembly 104. This in turn increases the accuracy of the pupil-tracking (as part of the eye-tracking) performed by an eye-tracking software program receiving the captured eye-tracking data.

The configuration illustrated in FIGS. 3A-C, in another aspect, provides a static reference point to track for head movement of the user. Specifically, with an infrared source (such as IR device 302) placed at or near the bottom center of monitor 301 and facing a subject eye, as illustrated in FIG. 3D, the infrared source creates a glint 313 outside pupil 311 and towards the bottom of eye 310. The created glint may be tracked to gain desirable tolerance for head movement.

In this embodiment, the bottom center location is chosen because such an arrangement results in the created glint being outside the pupil, a tracking type known as dark-pupil illumination. Compared to another tracking type known as light-pupil illumination referring to a created glint being inside the pupil, dark-pupil illumination has a notable advantage of creating a higher contrast between the usually dark pupil and the usually colored cornea, a scenario which enhances accuracy of tracking the created glint. In the eye-tracking environment exemplified in FIG. 3B, if IR device 302 were placed at the top of monitor 302, that arrangement would have created a glint in the center of the pupil, which is not optimal for glint-tracking.

Referring to FIG. 3D, the sufficient illumination on subject eye 310, as resulted from the configuration, renders the created glint 313 clearly visible and situated outside pupil 311, thus allowing the eye-tracking software program to optimally perform glint-tracking (in addition to pupil-tracking) as part of eye-tracking. In the conventional art, the infrared source that comes with a commercial eye-tracking product is usually arbitrarily placed only for the infrared illumination purpose. Often times, the infrared source may even be so placed that the infrared source moves as the human head moves or that results in a light-pupil illumination scenario which is undesirable for glint-tracking. Thus, in the conventional art, the position of an infrared source (such as IR device 302) relative to the position of the monitor (where cursor movements are displayed), even if provided, is usually arbitrary and vastly inaccurate, and not specifically calculated to generate a scenario optimal for glint-tracking. Hence, even if a glint is tracked for detecting head movement, the detection result is usually highly inaccurate and unreliable, and thus cannot be used to improve the quality of eye-tracking.

In contrast, with Applicant's bottom-center configuration, since IR device 302 is stationary relative to monitor 301 and the screen dimensions of monitor is usually known by the eye-tracking software program (receiving the eye-tracking data), the eye-tracking software program knows the position of IR device 302 relative to monitor 301 when, for example, the user informs the eye-tracking software program of the position of IR device 302 relative to the monitor. In one implementation, the user may inform the eye-tracking software program of the relative position of IR device 302 by configuring the eye-tracking software program via either a configuration file or a user interface letting user to customize various settings (including a setting defining IR device 302's relative position to monitor 301). Since the created glint 313 does not move in relation to monitor 301 when the user moves his/her head, the glint thus becomes a reliable static reference point to track for head movement. Also, as noted above, the bottom-center configuration results in a dark-pupil illumination scenario, which is optimal for tracking the created glint, and can generate accurate tracking results. Thus, with the bottom-center configuration, the eye-tracking software program is able to accurately track the created glint and use the tracking results to acquire accurate head movement information.

When able to acquire accurate head movement information (from tracking the glint) otherwise unavailable, the eye-tracking software program may in turn enhance the quality of eye-tracking by purposefully compensating any detected head movement in the midst of translating, for example, captured pupil-movement(s) to corresponding cursor movements, thereby realizing better HCI results.

A skilled artisan readily appreciates that various changes can be made to the configuration without departing from the scope and the spirit of the present disclosure. As one example, IR device 302 does not have to be attached to the bottom edge of monitor 301. IR device 301 may instead be disposed stand-alone and slightly away from the bottom center of monitor 301. As another example, IR device 302 may be positioned anywhere along the bottom edge of monitor 301, or anywhere that may generate a suitable glint-tracking scenario (such as a dark-pupil illumination scenario), as long as the positioning allows IR device 302 to provide adequate illumination on an subject eye 310 and the user may convey the information about the position of IR device 302 relative to monitor 301 to the eye-tracking software program performing the eye-tracking operation.

In summary, the wearable apparatus (including a headset 111 and an eye-tracking imaging assembly 104) and the relative positioning configuration exemplified in the figures achieves several advantages over conventional art. First, the illustrated headset 100 is easy to use while highly effective in terms of realizing selective and optimal positioning of an eye-tracking imaging assembly 104 close to the center of an eye for a clear view of the eye (including the pupil, the cornea and the glint). Such a realization of optimal positioning is advantageous for eye-tracking, since the captured eye-tracking data are usually of better quality, thus allowing the eye-tracking software program (receiving the better-quality eye-tracking data) to produce more accurate and effective HCI results.

Second, the illustrated headset 100 is also highly portable. As illustrated in FIGS. 1C and 1D, headset 100 is of relatively small size and can be folded or unfolded into dimensions suitable for storage and carriage.

Third, with the relative-positioning configuration illustrated in FIGS. 3A-D in conjunction with the eye-tracking imaging assembly 104 illustrated in FIG. 2, not only high-quality infrared illumination on an subject eye can be achieved, but also head movement can be accurately detected via tracking of a clearly visible and optimally situated glint resulted from the configuration. This combination in turn greatly enhances the quality of eye-tracking performed by an eye-tracking software program, thus resulting in HCI of better quality.

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular system, device or component thereof to the teachings of the disclosure without departing from the essential scope thereof.

Therefore, it is intended that the disclosure not be limited to the particular embodiments disclosed for carrying out this disclosure, but that the disclosure will include all embodiments falling within the scope of the appended claims.

1. A wearable apparatus adapted to be used for eye-tracking when coupled to a wearable eyewear frame, the apparatus comprising:
a coupling member structured and configured to securely couple the apparatus to the eyewear frame;
an arm assembly having a distal end, the arm assembly structured and configured to allow the distal end to move vertically and laterally; and
an imaging assembly configured to capture clear image data of an eye in close proximity and disposed on the distal end of the arm assembly such at vertical and lateral movements of the imaging assembly are realized by corresponding vertical and lateral movements of the distal end; and
wherein, as a user wears the eyewear frame with the coupling member coupled to the eyewear frame, the imaging assembly, through a combination of vertical and lateral movements thereof, is disposed in a position close to an eye of the user and suitable for eye-tracking.

2. The apparatus of claim 1, wherein the arm assembly is pivotably coupled to the coupling member such that vertical movements of the imaging assembly are realized by pivotal movements of the arm assembly.

3. The apparatus of claim 2, wherein the arm assembly comprises an arm member and a linkage member, one end of the arm member pivotably coupled to the coupling member, a non-pivotal end of the arm member joined to a proximal end of the linkage member to form an L-shape, and the distal end of the linkage member being the distal end of the arm assembly.

4. The apparatus of claim 3, wherein the distal end of the linkage member is retractable and extendable such that lateral movements of the imaging assembly are realized by retracting and extending the distal end of the linkage member.

5. The apparatus of claim 1, wherein the coupling member comprising a first side member and a second member, both configured to securely retain a part of the eyewear frame when the part of the eyewear frame is disposed there-between.

6. The apparatus of claim 1, wherein the imaging assembly is configured to wirelessly transmit captured image data to a receiving device.

7. The apparatus of claim 1, wherein the imaging assembly is configure to pass infrared light so as to capture moving images of an infrared-illuminated eye.

8. An imaging assembly used for eye-tracking, the imaging assembly comprising:
a lens module configured to allow clear focusing on an eye in close proximity;
an infrared pass filter module configured to allow infrared light to pass;
an imaging module configured to capture image data as a result of incoming light passing through the lens module and the infrared pass filter module; and
a wireless transmitter module configured to wirelessly transmit the captured image data to receiving device.

9. The imaging assembly of claim 8, wherein the imaging assembly is of a small size relative to a human face such that the imaging assembly is adapted to be suitably deployed on an apparatus coupled to an eyewear frame and disposed for eye-tracking in a position slightly below an eye of a user when the user wears the eyewear frame.

10. An eye-tracking system, the system comprising:
a monitor configured to display cursor movements;
an infrared light source disposed at a fixed location relative to the monitor;
an imaging assembly configured to pass infrared light and capture image data on a nearby human eye illuminated by infrared light, the imaging assembly disposed in a position close to an eye of a user and suitable for eye-tracking; and
a computing device configured to receive from the imaging assembly image data of eye movements, and perform an eye-tracking operation by translating the received image data into corresponding cursor movements on the monitor; and
wherein the infrared light source is so disposed that the eye of the user is adequately illuminated and a glint is created outside the pupil of the eye.

11. The system of claim 10, wherein the created glint is tracked so as to track and detect head movement of the user, and the detected head movement is compensated during the eye-tracking operation performed by the computing device.
